(19) United States
(12) Patent Application Publication
(10) Pub. No.: US 2009/0156930 A1
(43) Pub. Date: Jun. 18, 2009

(54) IMAGING SIMULATION FROM A REFERENCE TO A TILT ANGLE

(76) Inventor: Moshe Ein-Gal, Ramat Hasharon (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 11/954,534

(22) Filed: Dec. 12, 2007

(51) Int. Cl. A61B 5/05

(52) U.S. Cl. 600/425

(57) ABSTRACT

A method including obtaining reference image data-sets of a tissue related to respective reference patient tilt angles, wherein each reference image data-set is associated with a respective tilt angle, which is a combination of axial and transverse gravitational force components applied to the tissue, using the reference image data-sets to derive a formula relating an image data-set of said tissue to a given combination of axial and transverse gravitational force components, and using the formula to simulate an image data-set of a desired patient tilt angle.

### FIELD OF THE INVENTION

The present invention relates generally to medical imaging techniques, and particularly to methods for imaging simulation based on a reference image data-set to a desired patient tilt angle.

### BACKGROUND OF THE INVENTION

Treatment planning for radiotherapy makes use of CT images obtained with a recumbent patient. Imaging and treatment done in the same position assure positional consistency of the internal organs. However, although radiotherapy is usually applied to a recumbent patient, upright radiotherapy is also used. It logically follows that treatment planning should make use of CT images obtained with an upright patient. However, CT scanners are presently incapable of scanning an upright patient: they are tilt-limited (tilt range is about ±30°) and are not operable to tilt the patient.

Using recumbent imaging for upright treatment planning may give rise to imaging mismatches due to the respectively different gravitational forces applied to the patient during recumbent and upright imaging. The shape of anatomical or implanted markers is not generally affected, but soft-tissue organs may both deform and move.

While the mismatch might be minute, it may prevent precise target irradiation and increase radiation to adjacent organs. Prostate treatment, for example, is sensitive to such a mismatch due to the target's close proximity to radiation-sensitive organs like the rectal wall and the bladder.

Tissue deformation (and associated images) is related to the force field and the mechanical nature of the organs involved. The spatial distribution of tissue-dependent properties, e.g., Young's modulus and Poisson's ratio, affects tissue deformation. Various models are available for relating tissue deformation to a force field. Such models are typically used to estimate tissue deformation (and imaging thereof) given a force field, tissue mechanical properties and boundary conditions. It is also possible to solve a reverse problem, i.e., to determine the mechanical properties from given deformations, boundary values and a force field.

Since directly obtaining upright imaging is not feasible with present CT scanners, there is a need for simulating upright imaging for the purpose of upright treatment planning.

### SUMMARY OF THE INVENTION

The present invention seeks to provide novel methods for imaging simulation based on a reference image data-set to a desired patient tilt angle, as described more in detail hereinbelow.

There is thus provided in accordance with an embodiment of the present invention a method including obtaining reference image data-sets of a tissue related to respective reference patient tilt angles, wherein each reference image data-set is associated with a respective tilt angle, which is a combination of axial and transverse gravitational force components applied to the tissue using the reference image data-sets to derive a formula relating an image data-set of said tissue to a given combination of axial and transverse gravitational force components, and using the formula to simulate an image data-set of a desired patient tilt angle.

The formula may be derived by applying a selected bio-mechanical model relating a measured relative deformation and a force field associated with the reference data-sets in terms of tissue parameters related to mechanical properties of the tissue, and wherein simulating the image data-set of the desired patient tilt angle is performed by inserting the tissue parameters and a force field related to the desired patient tilt angle in the selected bio-mechanical model and using the formula to calculate the deformation.

For example, one of the reference image data-sets relates to a first patient tilt angle for a supine position of a patient and another reference image data-set relates to a second patient tilt angle for a prone position of the patient. As another example, one of the reference image data-sets relates to a first patient tilt angle of plus 30° and another reference image data-set relates to a second patient tilt angle of minus 30°. As yet another example, one of the reference patient tilt angles corresponds to a recumbent position and the desired patient tilt angle corresponds to an upright position.

In accordance with an embodiment of the present invention, the measurement includes forming a 3D contour of the tissue in the reference image data-sets and measuring the contour's variations in the data-sets.

Further in accordance with an embodiment of the present invention, the reference image data-sets are obtained while applying a pressure to the tissue.

There is also provided in accordance with an embodiment of the present invention a method for simulating desired tissue data-set related to a desired patient tilt angle including positioning a patient in a reference patient tilt angle, attaching a rigid container to a tissue in a known position relative to the patient, applying a force on the tissue such that the tissue adopts the shape of the container, obtaining a reference image data-set, and applying a formula to the reference image data-set to obtain the desired breast data-set related to a desired patient tilt angle.

There is also provided in accordance with an embodiment of the present invention a couch for scanning a tilted patient including a tabletop rotatable about a horizontal axis, a tilting device for tilting the tabletop to a desired patient tilt angle about the horizontal axis, and an actuator for moving the tabletop longitudinally perpendicular to the horizontal axis. The tabletop, the tilting device and the actuator may be constructed as an add-on device to an existing CT couch.

There is also provided in accordance with an embodiment of the present invention a container for partially enclosing therein a first body portion (e.g., a breast or abdomen), said container operable to keep the partially enclosed first body portion in a given position relative to a second body portion (e.g., the breast relative to the patient's chest, or the abdomen relative to the patient's pelvis) irrespective of tilt-angle, wherein the first body portion is forced to comply with the internal shape of the container, and wherein the container is constructed to enable imaging of said first body portion (for example, using X-rays, ultrasonic waves, magnetic resonance, etc.).

### BRIEF DESCRIPTION OF THE DRAWINGS

The present invention will be understood and appreciated more fully from the following detailed description, taken in conjunction with the drawings in which:

FIG. 1 is a simplified flow chart of a method for imaging simulation based on a reference image data-set to a desired patient tilt angle, in accordance with an embodiment of the present invention;

FIG. 2 is a simplified illustration of apparatus for imaging a breast, constructed and operative in accordance with an embodiment of the present invention; and

FIG. 3 is a simplified illustration of a couch for scanning a tilted patient, constructed and operative in accordance with an embodiment of the present invention.

### DETAILED DESCRIPTION OF EMBODIMENTS

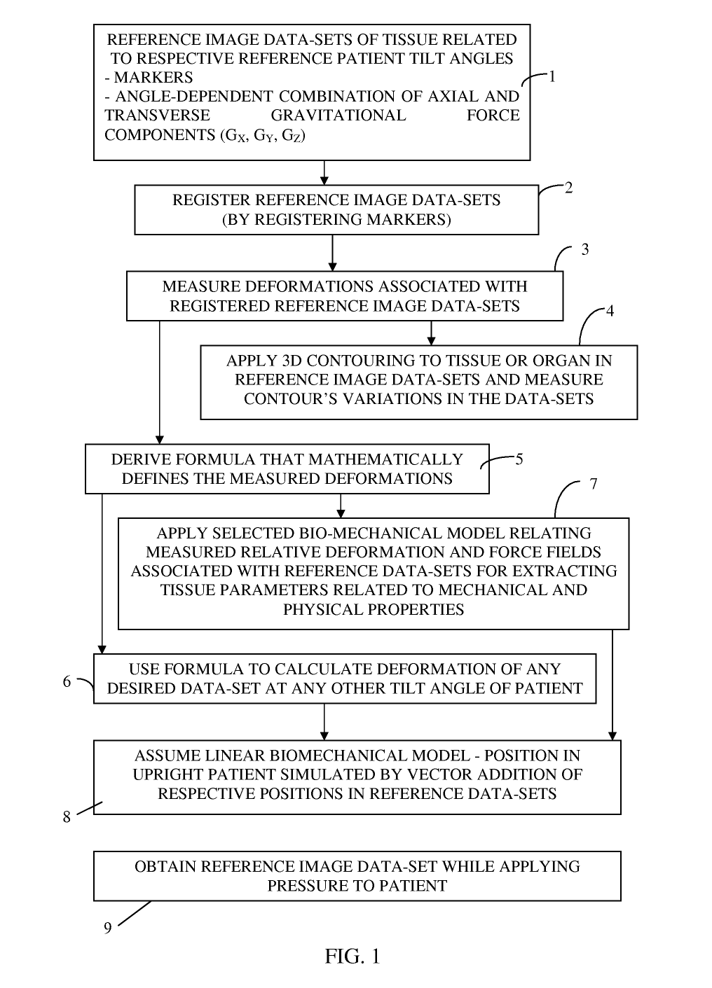

Reference is now made to FIG. 1, which is a simplified flow chart of a method for imaging simulation based on a reference image data-set to a desired patient tilt angle, in accordance with an embodiment of the present invention.

The method makes use of reference image data-sets of a tissue or group of tissues, related to respective reference patient tilt angles (1). The reference image data-sets may be obtained with any suitable imaging method, such as but not limited to, ultrasound (US), computed tomography (CT), single photon emission computed tomography (SPECT), magnetic resonance imaging (MRI), magnetic resonance spectroscopy (MRS), positron emission tomography (PET), etc. Each reference image data-set is associated with an angle-dependent combination of axial and transverse gravitational force components (Gₓ, G_{y}, G_{z}), which are representative of the droop, sag or other deformation at the particular tilt angle. The reference angles of the reference image data-set may encompass any patient orientation, such as but not limited to, supine, prone, ±30° and many others.

The method of the invention can use more than one reference image data-set. In such a case, the reference image data-sets must be registered (2). The registration can be accomplished by using markers in the reference image data-sets and causing the markers to coincide (i.e., registering them).

The tissue or group of tissues is then tilted at an angle. The gravity force field applied to the tissue(s) causes deformation of the tissue(s). The deformations associated with the registered reference image data-sets are measured (3). For example, the measurement may include applying 3D contouring to an organ in the reference image data-sets and measuring the contour's variations in the data-sets (4).

A formula (mathematical function) is then derived that mathematically defines the measured deformations (5). This formula can then be used to calculate the deformation of any desired data-set at any other tilt angle of the patient (6), such as in an upright position.

One way of deriving the formula is to apply a selected bio-mechanical model relating the measured relative deformation and the force fields associated with the reference data-sets for extracting tissue parameters related to mechanical and physical properties (elasticity modulus, tensile strength, etc.) (7). Simulating an image data-set of a desired patient tilt angle is then performed by inserting the extracted tissue parameters and a force field related to the desired patient tilt angle in the selected bio-mechanical model.

It is noted, that if the first reference patient tilt angle is 30° and the second reference patient tilt angle is −30°, then the combined gravitational forces acting on the patient in the two tilt angles equal a gravitational force acting on an upright patient. Assuming a linear biomechanical model, a point's position in the upright patient can be simulated by vector addition of the point's respective positions in the two reference data-sets (8).

Many other mathematical techniques can be used to derive the formula (e.g., least squares, weighted averages, etc.).

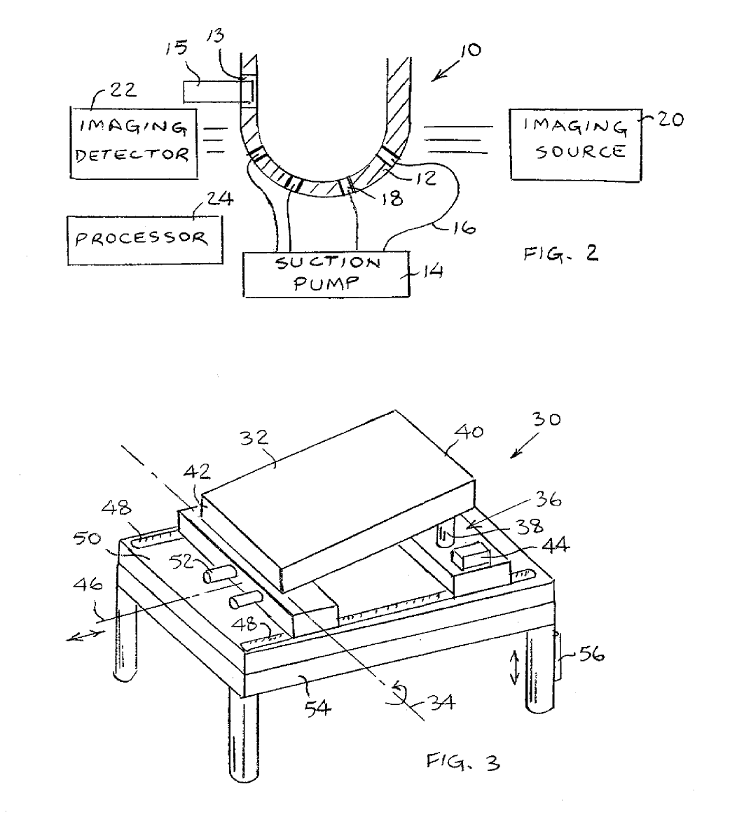

In accordance with another embodiment of the invention, the reference image data-set may be obtained while applying a pressure to the patient (9). The external pressure, added to the gravitational force, changes the ratio of axial and transversal force components, thus providing an additional independent measurement. The external pressure may be positive, e.g., when applied to the abdomen by a pressurized belt or to the breast by a container forcing the breast to retain its shape for various patient tilt angles. The pressure can also be negative, e.g., when a vacuum is applied to a container containing the breast. An embodiment that applies pressure to a breast for imaging thereof is described now with reference to FIG. 2.

FIG. 2 illustrates apparatus 10 for imaging a breast or procedures such as biopsy, excision, RF ablation or radiation treatment, in accordance with an embodiment of the present invention.

Apparatus 10 includes a rigid, generally radiation-transparent container 12 for containing and fixing a breast therein. Container 12 is arranged such that a force can be applied on the breast so that the breast adopts the shape of container 12. For example, the force on the breast may be applied manually, by gravitation (lying face down), or by applying a vacuum from a suction pump 14 connected via tubing 16 to a plurality of apertures 18 formed in container 12, or any combination thereof. The force is sufficient to compress the breast to become generally rigid inside container 12. Imaging apparatus (e.g., a CT scanner), comprising an imaging source 20 and an imaging detector 22, are provided for imaging the breast as held in container 12. Container 12 may have a window 13 allowing a contact of an imaging or a procedure device 15 with the breast.

The apparatus 10 of FIG. 2 may be used to carry out the method of FIG. 1. Accordingly, the patient is positioned in a reference patient tilt angle. The rigid container 12 is attached in a known position relative to the patient, and a force is applied on the breast such that the breast adopts the shape of container 12 (the apparatus 10 could also be configured for other body portions, such as but not limited to, the abdomen). The internal shape of container 12 causes the compressed breast to extend longitudinally and be compressed radially. The imaging apparatus captures images of the breast to create one or more reference image data-sets (as in step 1 of FIG. 1). Image data-sets are registered (as in step 2 of FIG. 1) and any deformation is measured (as in step 3 of FIG. 1). A formula is obtained that mathematically defines the deformation (as in step 5 of FIG. 1). The formula is used to calculate the deformation of any desired data-set at any other tilt angle of the patient (as in step 6 of FIG. 1), such as in an upright position. A processor 24 may be provided for calculating the formula and any data processing, and for simulating a desired data-set related to a desired patient tilt angle.

Forcing the breast into a rigid container reduces the breast's orientation-dependent deformation, similar to the soft brain inside the rigid cranium. Since the breast tissue rigidity increases with compression, the container volume and the applied force should be, respectively, small enough and large enough to cause breast rigidity. In the event the breast is sufficiently rigid and the desired data-set relates to the breast contained in a rigid container, no application of a formula may be required and the reference image data set may be used as the desired data-set.

Breast CT scanning of a supine patient, e.g., for radiation treatment planning, causes an undesirable proximity of the breast to the chest wall. In mammography, the breast of an upright patient is radially compressed between two parallel plates, causing the breast to mainly extend in a perpendicular radial direction. The embodiment of FIG. 2 causes the breast to generally extend longitudinally—thus separating the breast from the chest-wall, while being compressed radially—thus increasing the breast's rigidity and shape retention. For obtaining a reference CT data-set related to the breast of a supine patient, vacuum may be applied to pull the breast away from the chest-wall: the vacuum level should be high enough to combat gravitation. During treatment of the upright patient, the vacuum level may be reduced.

Accordingly, in accordance with an embodiment of the present invention the object to be imaged is pressurized and encapsulated in a rigid and radiation transparent enclosure such that imaging differences between recumbent and upright positions are minimized or negligible. (This, for example, is naturally the case with the brain inside the skull). Such an encapsulation may have imaging-sensitive markings for determining internal organ positions relative to the external encapsulation. Such an encapsulation may be used for immobilizing the pelvis area for imaging and treatment, e.g., the prostate, in a generally upright position. The pelvis encapsulation will have four tight “walls” and a bottom made of an elongated “seat” pressing against the pubic bone between the legs (like a bicycle seat). The encapsulation may be tightened to the pubic bone by straps over the shoulders. Following imaging with the encapsulation, the target position relative to the encapsulation is determined (as described above with reference to FIG. 1) and the encapsulation will be accordingly positioned for treatment.

Reference is now made to FIG. 3, which illustrates a couch 30 for scanning a tilted patient, constructed and operative in accordance with an embodiment of the present invention. Couch 30 includes a tabletop 32 rotatable about a horizontal pivot axis 34 by means of a tilting device 36. In accordance with one non-limiting embodiment of the invention, tilting device 36 includes a jack 38 (electrical, mechanical, hydraulic or pneumatic) that lifts or lowers one end 40 of tabletop 32, thereby tilting or pivoting the other end 42 of tabletop 32 about pivot axis 34 to a desired patient tilt angle. A controller 44 may be provided that controls operation of jack 38.

Tabletop 32 is preferably movable along a longitudinal axis 46. For example, tabletop 32, with or without the tilting device 36, may slide along one or more tracks 48 formed in a base 50. One or more linear actuators 52 coupled to tabletop 32 may move tabletop 32 in the longitudinal (horizontal) direction along tracks 48. Controller 44 may also control the operation of linear actuators 52.

Most CT scanners perform helical scans by rotating the gantry about the recumbent patient while moving the patient axially during scanning. In the present invention, for scanning a tilted patient, the scanner is tilted with a similar tilt angle. A patient tilt angle larger than a scanner-tilt angle can be used with scanners not employing axial patient motion during scanning.

The couch 30 of FIG. 3 may be constructed as a standalone couch system. Alternatively, it may be constructed as an add-on device to an existing CT couch 54. In any case, another actuator 56 may be provided for moving the couch in a vertical direction. The controller 44 may control the CT couch vertical motion as well, such that the combined horizontal and vertical motions of the couch are generally along the tilted additional tabletop 32.

The scope of the present invention includes both combinations and subcombinations of the features described hereinabove as well as modifications and variations thereof which would occur to a person of skill in the art upon reading the foregoing description and which are not in the prior art.

What is claimed is:

1. A method comprising:
obtaining reference image data-sets of a tissue related to respective reference patient tilt angles, wherein each reference image data-set is associated with a respective tilt angle, which is a combination of axial and transverse gravitational force components applied to the tissue;
using the reference image data-sets to derive a formula relating an image data-set of said tissue to a given combination of axial and transverse gravitational force components; and
using the formula to simulate an image data-set of a desired patient tilt angle.

2. The method according to claim 1, wherein the formula is derived by applying a selected bio-mechanical model relating a measured relative deformation and a force field associated with the reference data-sets in terms of tissue parameters related to mechanical properties of the tissue, and wherein simulating the image data-set of the desired patient tilt angle is performed by inserting said tissue parameters and a force field related to the desired patient tilt angle in the selected bio-mechanical model and using said formula to calculate the deformation.

3. The method according to claim 1, wherein one of the reference image data-sets relates to a first patient tilt angle for a supine position of a patient and another reference image data-set relates to a second patient tilt angle for a prone position of the patient.

4. The method according to claim 1, wherein one of the reference image data-sets relates to a first patient tilt angle of plus 30° and another reference image data-set relates to a second patient tilt angle of minus 30°.

5. The method according to claim 1, wherein the measurement includes forming a 3D contour of the tissue in the reference image data-sets and measuring the contour's variations in the data-sets.

6. The method according to claim 1, wherein one of the reference patient tilt angles corresponds to a recumbent position and the desired patient tilt angle corresponds to an upright position.

7. The method according to claim 1, wherein the reference image data-sets are obtained while applying a pressure to the tissue.

8. A method for simulating desired tissue data-set related to a desired patient tilt angle comprising:
positioning a patient in a reference patient tilt angle;
attaching a rigid container to a tissue in a known position relative to the patient;
applying a force on the tissue such that the tissue adopts the shape of the container;
obtaining a reference image data-set; and
applying a formula to the reference image data-set to obtain the desired tissue data-set related to a desired patient tilt angle.

10. A couch for scanning a tilted patient comprising:
a tabletop rotatable about a horizontal axis;
a tilting device for tilting said tabletop to a desired patient tilt angle about the horizontal axis; and
an actuator for moving said tabletop longitudinally perpendicular to the horizontal axis.

11. The couch according to claim 10, wherein said tabletop, said tilting device and said actuator comprise an add-on device to an existing CT couch.

12. Apparatus comprising:
a container for partially enclosing therein a first body portion, said container operable to keep said first body portion in a given position relative to a second body portion irrespective of tilt-angle, wherein said first body portion is forced to comply with an internal shape of said container, and wherein said container is constructed to enable imaging of said first body portion.
